(19) United States
(12) Patent Application Publication
(10) Pub. No.: US 2004/0247535 A1
(43) Pub. Date: Dec. 9, 2004

(54) Oral composition for treating sensitive teeth

(76) Inventors: Taek-Kyun Kang, Daejeon (KR); Young-Ho Kim, Daejeon (KR); Jae-Hyun Ahn, Daejeon (KR)

Correspondence Address: VENABLE, BAETJER, HOWARD AND CIVILETTI, LLP, P.O. BOX 34385, WASHINGTON, DC, 20043-9998, US

(21) Appl. No.: 10/491,109

(22) PCT Filed: Sep. 18, 2002
(86) PCT No.: PCT/KR02/01748

(30) Foreign Application Priority Data
Sep. 27, 2001 (KR) 2001/59880

(51) Int. Cl. A61K 7/16

(52) U.S. Cl. 424/57

(57) ABSTRACT

The present invention relates to an oral composition which may prevent or treat sensitive teeth. The oral composition contains potassium phosphates, wherein potassium ions inactivate dental nerves to relieve temporary pains, and phosphates not only prevent the exposure of dentinal tubules, which is the basic cause of sensitive teeth, but also block the exposed dentinal tubules. The oral composition further comprises ursodesoxycholic acid, thereby improving the effect of relieving the pain caused by sensitive teeth.

### TECHNICAL FIELD

The present invention relates to an oral composition which may prevent and treat sensitive teeth.

### BACKGROUND ART

In sensitive teeth, the dentin layer in a tooth is exposed through the abrasion or damage of enamel or exposure of root surface which is caused by an innate gingival defect, wrong method of brushing teeth, or use of toothpaste of strong abrasiveness.

When the exposed dentin layer is touched with external stimuli such as mechanical or chemical stimuli, hot or cold fluids, the one with the sensitive teeth feels chilly discomforts and even an acute pain.

The dentin hypersensitivity comes from the dentinal tubules formed in the dentin. When the dentin layer is covered with enamel layer in a normal condition, nerve cells located in the base of dentinal tubules are separated from the outside so that changes in outside circumstance cannot be transferred to the nerve cells. While the enamel layer is abraded by the above-mentioned reasons and the dentinal tubules are exposed, then the external stimuli may be transferred to the nerve cells through the dentinal tubules.

There are many conventional treatments of sensitive teeth which include the following two methods.

The first is blocking or plugging the exposed dentinal tubules. In U.S. Pat. No. 5,211,939 organic materials such as Carbopol or polystyrene beads are used, and U.S. Pat. Nos. 4,634,589 and 4,710,372 describes hydroxyapatite. Further, mineral materials such as montmorrolinite clay are also used in U.S. Pat. No. 4,992,258. The particulates of these materials enter into the dentinal tubules and plug the exposed tubules to block the transmission of external stimuli into the nerve cells. Alternately, it is suggested that a mineral precipitation is formed on the surface of the dentinal tubules without directly adding particles into the tubules. An example of the mineral precipitation is made of ferric oxalate or potassium oxalate.

The second is disturbing the electrolyte balance around the nerves to prevent the excitement of the nerves even with the stimuli coming through the dentinal tubules, thereby weakening the pain derived from the equal degree of stimuli. Alkali metal ions are generally used for this purpose. Potassium nitrate in U.S. Pat. No. 3,863,006 and potassium hydrogen carbonate in U.S. Pat. No. 4,631,185 are disclosed to be used for relieving the hypersensitivity. Many other oral compositions containing potassium salts are suggested. In U.S. Pat. No. 3,122,483, strontium chloride is used instead of potassium ions.

Besides, potassium hydroxide, magnesium hydroxide, sodium chloride, silver nitrate, sodium citrate and other salts are also used for relieving hypersensitivity. It is believed that these salts contained in dentifrices are absorbed into the exposed dentin to disturb the ion balance around the nerve fibers, thereby relieving the hypersensitivity.

Especially, according to the clinical research by John W. Peden in The Journal of the Western Society of Periodontology, 25:2 (Summer 1977), the literature of Barry Lee Green, et al. in Journal of Periodontology, 48:10 (October 1977), the clinical research by Willard J. Tarbet, et al. in Journal of Periodontology, 51:9 (September 1980) and the clinical research by Milton Hodosh, in Journal of American Dental Association, 88 (April 1974), potassium nitrate is described as the most effective among such agents as typically used in relieving hypersensitivity. It is known that the relieving effect of potassium nitrate is due to potassium ion.

The first method described above is an active solution to treat sensitive teeth by plugging the exposed dentinal tubules, while the second is a passive makeshift to get a temporary relief of pain. Nonetheless, products according to the first method are rarely available, while the dentifrices containing potassium ion such as potassium nitrate or potassium chloride are mostly developed.

### DISCLOSURE OF THE INVENTION

It is an object of the present invention to provide an oral composition for sensitive teeth which may inhibit the transmission of external stimuli by blocking the dentinal tubules as well as relieve the pain through the alleviation of excitement of nerves by potassium ion.

In accordance with one aspect of the present invention, it is provided an oral composition for sensitive teeth comprising a potassium phosphate, wherein the amount of potassium ranges from 0.4 to 4% by weight of the oral composition in the oral composition of the present invention containing a potassium phosphate, the phosphate, which are a constituent of teeth, enhances the remineralization of the dentinal tubules to fill and plug them. Further, the isolated potassium ion of the potassium phosphate changes the ion balance around the nerves to work for inactivating the nerves. Preferably, the oral composition further comprises medicinal components for preventing the gum recession, which also prevents the exposure of dentin.

The oral composition of the present invention contains a potassium phosphate which may provide phosphate, a component of hydroxyapatite which is a constituent of teeth, and also relieve the pain by changing the ion balance between potassium and sodium. Representative examples of the potassium phosphate are tribasic potassium; phosphate (K₃PO₄), dibasic potassium phosphate (K₂HPO₄) and monobasic potassium phosphate (KH₂PO₄). Further, the oral composition of the present invention may include ursodesoxycholic acid which is effective against the gum recession.

The oral composition of the present invention may be provided in the typical tubes for paste as the end preparation in package form. In the oral composition of the present invention, the potassium phosphate is included in an amount ranging from 0.4 to 4 g measured in terms of total potassium ion in 100 g of the composition, which is equivalent to the amount ranging from 1 to 10 g of potassium nitrate in 100 g of conventional toothpaste. The amount of less than the minimum value has little, effect. If the amount is more than the maximum value., the rise in cost exceeds the rise in effect and the stability of the composition decreases. Preferably, the amount of potassium in the potassium phosphate ranges from 1 to 4%, based on the weight of the composition.

In the oral composition of the present invention, ursodesoxycholic acid may be added in an amount of 0.01 to 1 g in 100 g of the composition. If the amount is less than the minimum value, the composition has little effect. If the amount is more than the maximum value, the rise in cost exceeds the rise in effect.

In addition to the effect on relieving the hypersensitivity of teeth, the oral composition according to the present invention has an effect on alleviating the inflammation of gums and preventing its recession by weakening the activity of collagenase, which is due to ursodesoxycholic acid. Accordingly, the oral composition of the present invention enhances the remineralization of teeth to block the dentinal tubules as well as relieves the disease of gums to prevent the exposure of dentinal tubules.

The oral composition of the present invention may comprise an abrasive. The abrasive which may be used in the present invention is precipitated silica, silica gel, zirconium silicate, dicalcium phosphate dihydrate, anhydrous dicalcium phosphate, hydrated alumina, calcium carbonate, calcium pyrophosphate, insoluble metaphosphate, aluminum silicate or mixtures thereof. The abrasive is added in an amount of 5 to 60% by weight of the composition, so that the abrasiveness (RDA) of the final product may have the value ranging from 20 to 100. The size of abrasive varies depending on types, and that of mean particle diameter less than 20 μm is preferred to be used.

As to the abrasiveness of toothpaste on the market, an abrasiveness of 50 or less is typically regarded as low-abrasiveness, the value of 50 to 100 belongs to middle-abrasiveness, and that of 100 or more is high-abrasiveness. Using a toothpaste having the abrasiveness of 100 or more for a long period may provoke abrasion of root surface to aggravate the hypersensitivity of teeth. Considering this, it is preferred to use a toothpaste of low abrasiveness. However, a toothpaste having the abrasiveness of 30 or less has little effect on removing plaque, so that it may make the oral condition worse.

A humectant, which is required to make an paste preparation, prevents toothpaste from drying and solidifying when exposed to air and provides gloss to the surface of toothpaste. Further, some humectants give a sweet taste at the time of brushing. The humectant which may be used in the oral composition of the present invention is a concentrated glycerin (98%), glycerin (85%), a sorbitol solution (70%), xylitol, polyethylene glycols, propylene glycol, or mixtures thereof. The humectant is added in an amount ranging from 20 to 70% by weight of the oral composition.

Like many conventional toothpastes, the oral composition of the present invention may include a suitable medicinal component for some purpose. For example, fluoride compounds are, necessary to form a fluoride coating on teeth against some acids (e.g., lactic acid) produced by bacteria which induce dental caries. In order to prevent teeth and gums diseases, various vitamins, allantoin or allantoin derivatives, tranexamic acid, or aminocaproic acid, which has a hemostatic effect, may be used in the oral composition of the present invention. These medicinal components help the action of ursodesoxycholic acid against the gum recession, which makes an synergistic effect.

A binder, which prevents the separation of powder components and liquid, components, is also required in the oral composition of paste type. Any water-soluble polymer may be used as a binder of toothpaste. Examples of the binder are sodium carboxymethylcellulose derived from cellulose, carrageenans extracted from seaweeds, and xanthan gum obtained from metabolites of microorganism.

A preservative prevents the oral composition from being contaminated with microorganisms and helps to lengthen the storage life of the oral composition. Representative examples of the preservative are sodium benzoate and parabens.

Further, a foaming agent is used in the oral composition of the present invention for the purpose of producing a favorable impression on users, enhancing the cleaning effect, promoting the dispersion and penetration of the main component, and reducing the surface tension so that extraneous materials may be detached from the oral cavity. An anionic surfactant, sodium laurylsulfate is generally used as the foaming agent and such non-ionic surfactant as polyoxyethylene-polyoxypropylene copolymer (Poloxamer), polyoxyethylene-hydrogenated castor oil or polyoxyethylene sorbitan fatty acid ester is also used to supplement the function of anionic surfactant depending on preparations.

The oral composition of the present invention may include a flavor, a sweetener, and a coloring agent for better taste. For this purpose, it is essential to use a flavor for food. Examples of the flavor are peppermint oil, spearmint oil, sage, eucalyptol, methyl salicylate and an extract of fruits. Saccharin sodium is mostly used as a sweetener, and a food coloring is typically included.

### BRIEF DESCRIPTION OF THE DRAWINGS

The above and other objects, features and other advantages of the present invention will be more clearly understood from the following detailed description taken in conjunction with the accompanying drawings, in which:

FIG. 1A is a photograph of electron microscope showing the dentinal tubules treated by the oral composition prepared in Comparative Example 1;

FIG. 1B is a photograph of electron microscope showing the dentinal tubules treated by the oral composition prepared in Comparative Example 3; and

FIG. 1C is a photograph of electron microscope showing the dentinal tubules treated by the oral composition prepared in Example 4.

### BEST MODE FOR CARRYING OUT THE INVENTION

Hereinafter, the present invention will be described in detail, in conjunction with various examples. These examples are provided only for illustrative purposes, and the present invention is not to be construed as being limited to these examples.

In the Examples and Comparative Examples, an abrasive was silica, a humectant was sorbitol solution (70%) and polyethylene glycol, a binder was carboxymethylcellulose, a sweetener was saccharin, a foaming agent was sodium laurylsulfate, and a preservative was sodium benzoate, all of which were added to each Examples and Comparative Examples in the same amount. However, in case other components or other amounts than the above-mentioned were added to the Examples and Comparative Examples, there was no differences in the effects obtained therefrom.

### EXAMPLES 1 TO 6 AND COMPARATIVE EXAMPLES 1 TO 3

The following Table 1 shows each component and its amount to be used in the oral composition of Examples 1 to 6 and Comparative Examples 1 to 3. In Table 1, the unit of each value is % by weight.

TABLE 1

| | | | | | | | | Comparative | | |
|---|---|---|---|---|---|---|---|---|---|---|
| | | Examples | | | | | | Examples | | |
| | Components | 1 | 2 | 3 | 4 | 5 | 6 | 1 | 2 | 3 |
| Abrasive | (a) | 20 | 20 | 20 | 20 | 20 | 20 | 20 | 20 | 20 |
| Humectant | (b) | 50 | 50 | 50 | 50 | 50 | 50 | 50 | 50 | 50 |
| | (c) | 3 | 3 | 3 | 3 | 3 | 3 | 3 | 3 | 3 |
| Foaming | (d) | 2 | 2 | 2 | 2 | 2 | 2 | 2 | 2 | 2 |
| agent | | | | | | | | | | |
| Binder | (e) | 0.8 | 0.8 | 0.8 | 0.8 | 0.8 | 0.8 | 0.8 | 0.8 | 0.8 |
| Medicinal | (f) | 0.22 | 0.22 | 0.22 | 0.22 | 0.22 | 0.22 | 0.22 | 0.22 | 0.22 |
| components | (g) | 6.7 | — | — | 2 | 3 | 1 | — | — | — |
| | (h) | — | 4.3 | — | 3 | — | 2 | — | — | — |
| | (i) | — | — | 3.5 | — | 2 | 1.5 | — | — | — |
| | (j) | — | — | — | — | — | — | 5 | — | — |
| | (k) | — | — | — | — | — | — | — | 3.7 | — |
| Additives | (l) | 1 | 1 | 1 | 1 | 1 | 1 | 1 | 1 | 1 |
| | (m) | 0.25 | 0.25 | 0.25 | 0.25 | 0.25 | 0.25 | 0.25 | 0.25 | 0.25 |

| | | | | | | | | | |
|---|---|---|---|---|---|---|---|---|---|
| Purified water | 16.03 | 18.43 | 19.23 | 17.73 | 17.73 | 18.23 | 17.73 | 19.03 | 22.73 |

(a) precipitated silica
(b) non-crystalline sorbitol solution
(c) polyethylene glycol
(d) sodium laurylsulfate
(e) carboxymethyl cellulose
(f) sodium fluoride
(g) monobasic potassium phosphate
(h) dibasic potassium phosphate
(i) tribasic potassium phosphate
(j) potassium nitrate
(k) potassium chloride
(l) flavor
(m) saccharin

In each Examples and Comparative Examples, the toothpaste was prepared as follows:

(1) A binder and additives were dispersed in a humectant, an abrasive and medicinal components were added thereto, and then the mixture was stirred for 30 minutes; and

(2) A foaming agent was added to the mixture, which was stirred under a vacuum condition for 20 minutes to obtain an end product.

Efficiency Tests

(1) Test for Blocking of Dentinal Tubules in Human Teeth

The effects of oral compositions produced in Examples and Comparative Examples on blocking the dentinal tubules were tested according to the following steps:

\mathring{1} Each of the oral compositions of Examples 1 to 6 and Comparative Examples 1 to 3 was mixed with saliva (saliva:toothpaste=1:2) to make slurry, which was then put in a container placed on a magnetic stirrer.

\mathring{2} A tooth sample extracted from a healthy person in the twenties or thirties was fixed with epoxy resin, and then treated with acid so that the tooth enamel would be removed completely.

\mathring{3} The tooth sample was put into the slurry of step \mathring{1} and then stirred for 3 days. After then, each tooth sample was examined with an electron microscope (SEM).

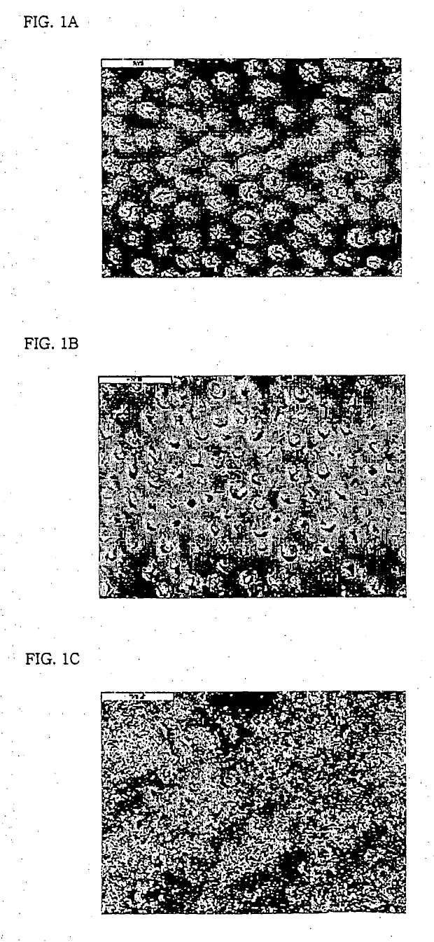

Each of the results obtained from Examples 1 to 6 shows no distinguished differences between them. Therefore, the representative result of Example 4 is compared with those of Comparative Examples 1 and 3. FIGS. 1A, 1B and 1C are photographs of electron microscope showing the dentinal tubules treated by the oral compositions prepared in Comparative Example 1, Comparative Example 3, and Example 4, respectively. As shown in FIGS. 1A to 1C, the oral composition according to the present invention has superior effect on blocking the dentinal tubules compared to those of Comparative Examples.

(2) Measure of Dentin Permeability

The test was carried out for comparing quantitatively the effect on blocking the dentinal tubules. Based on the process of Pashley (J. Periodontology, Vol. 55, No. 9, p552, September 1984), a fluid flow passed through the dentin disk was measured. Reduced flow means that the dentinal tubules have been blocked. The test was carried out according to the following steps:

\mathring{1} compositions of Examples 1 to 6 and Comparative Examples 1 to 3 was mixed with saliva (saliva:toothpaste=1:1) to make slurry.

\mathring{2} A tooth sample extracted from a healthy person in the twenties or thirties was cut into dentin disks of 0.4˜0.6 mm.

\mathring{3} Each disk were put in a separated chamber instrument (J. Dent. Research 57: 187, 178).

\mathring{4} The first fluid flow passed through the disk was measured, and then the disk was brushed with each slurry of step \mathring{1}.

\mathring{5} After brushing, the fluid flow passed through the disk was measured, and then decreased fluid was calculated.

Table 2 shows the results of the above test.

TABLE 2

| Samples | | Change of flow (%) |
|---|---|---|
| Examples | 1 | −63 |
| | 2 | −67 |
| | 3 | −67 |
| | 4 | −82 |
| | 5 | −79 |
| | 6 | −75 |
| Comparative Examples | 1 | −43 |
| | 2 | −36 |
| | 3 | −25 |

As shown in Table 2, the oral compositions according to the present invention have superior effect on blocking the dentinal tubules compared with those of Comparative Examples.

### EXAMPLES 7 TO 9

The oral compositions of Examples 7 to 9 were prepared by adding ursodesoxycholic acid in the amount of 0.02% by weight to those of Examples 4 to 6. The following table 3 shows each component and its amount to be used in the oral composition of Examples 7 to 9. In Table 3, the unit of each value is % by weight.

TABLE 3

| | | Examples | | |
|---|---|---|---|---|
| | Components | 7 | 8 | 9 |
| Abrasive | Precipitated silica | 20 | 20 | 20 |
| Humectant | Non-crystalline sorbitol solution | 50 | 50 | 50 |
| | Polyethylene glycol | 3 | 3 | 3 |
| Foaming agent | Sodium laurylsulfate | 2 | 2 | 2 |
| Binder | Carboxymethyl cellulose | 0.8 | 0.8 | 0.8 |
| Medicinal | Sodium fluoride | 0.22 | 0.22 | 0.22 |
| Components | Monobasic potassium phosphate | 2 | 3 | 1 |
| | Dibasic potassium phosphate | 3 | — | 2 |
| | Tribasic potassium phosphate | — | 2 | 1.5 |
| | Ursodesoxycholic acid | 0.02 | 0.02 | 0.02 |
| Additives | Flavor | 1 | 1 | 1 |
| | Saccharin | 0.25 | 0.25 | 0.25 |

| | | | |
|---|---|---|---|
| Purified water | 17.71 | 17.71 | 18.21 |

Table 4 shows the results of measuring the fluid flow of the oral compositions of Examples 7 to 9 passed through the dentin disks. As shown in Table 4, there is no difference in change of flow due to the addition of ursodesoxycholic acid.

TABLE 4

| Samples | | Change of flow (%) |
|---|---|---|
| Examples | 7 | −82 |
| | 8 | −79 |
| | 9 | −75 |

(3) Clinical Testing of the Relief of Hypersensitivity

The oral compositions prepared in Examples 4 and 7, and Comparative Examples 1 and 3 were used by candidates for 1 month, and the effects of the compositions on relieving the hypersensitivity were evaluated.

Those who have sensitive teeth were selected and their sensitive teeth were grouped. Before starting the test, the sensitive teeth were exposed to cold air and grouped so that the number of sensitive teeth and the extent of hypersensitivity would be about the same, in which the extent of hypersensitivity was evaluated on the basis of the criteria shown in Table 5.

TABLE 5

| Symptoms of hypersensitivity | Grades |
|---|---|
| No chilly discomfort | 1 |
| A little chilly discomfort but not sensible without attention | 2 |
| A little chilly discomfort and sensible without attention | 3 |
| A severe chilly discomfort but tolerable pain | 4 |
| A severe chilly discomfort and intolerable pain | 5 |

Each of the oral compositions was given to each group for being used. After two and four weeks of using the oral compositions, the extent of hypersensitivity was evaluated in the same manner. In this test, each group had forty sensitive teeth. Table 6 shows the results of the above clinical testing.

TABLE 6

| | Extent of hypersensitivity | | |
|---|---|---|---|
| Oral Composition | Before starting | After 2 weeks | After 4 weeks |
| Example 4 | 3.21 ± 0.88 | 2.73 ± 1.08* | 2.27 ± 0.82** |
| Example 7 | 3.24 ± 0.91 | 2.71 ± 1.04* | 2.18 ± 0.76** |
| Comparative | 3.23 ± 1.18 | 2.74 ± 1.15 | 2.33 ± 1.00** |
| Example 1 | | | |
| Comparative | 3.31 ± 0.86 | 3.13 ± 1.07 | 3.08 ± 0.97 |
| Example 3 | | | |

* and ** mean that the values are significantly different at p < 0.05 and p < 0.01, respectively.

As noticed in the above results, the oral composition of the present invention showed the effect on relieving the hypersensitivity, which was not shown in that of Comparative Example 3. The relieving effect of the present invention was taken as fast as that of Comparative Example 1 which contained potassium nitrate, the representative component to relieve hypersensitivity. Especially, the oral composition of Example 7 has an effect better than that of Example 4. Further, the oral composition further containing ursodesoxycholic acid prevented the gum recession and showed a relieving effect better than that containing potassium nitrate only.

As described above, the effect of potassium phosphates on relieving the hypersensitivity has been confirmed. Further, the addition of ursodesoxycholic acid which prevents the gum recession can enhance the effect on relieving the hypersensitivity.

### INDUSTRIAL APPLICABILITY

As apparent from the above description, the oral composition according to the present invention, which contains potassium phosphates, relieves the temporary pain by the action of potassium ions, prevents the exposure of dentinal tubules, which is the root cause of sensitive teeth, and blocks the exposed dentinal tubules through the supply of phosphates, main components of teeth. The oral composition of the present invention may further comprises ursodesoxycholic acid in order to prevent the gum recession and enhance the effect on relieving the hypersensitivity.

What is claimed is:

1. An oral composition for sensitive teeth comprising a potassium phosphate, wherein the amount of potassium ranges from 0.4 to 4% by weight of the oral composition.

2. The oral composition for sensitive teeth of claim 1, wherein the amount of potassium in the potassium phosphate is 1 to 4% by weight of the oral composition.

3. The oral composition for sensitive teeth of claim 1 or 2, wherein the potassium phosphate is tribasic potassium phosphate (K₃PO₄), dibasic potassium phosphate (K₂HPO₄), monobasic potassium phosphate (KH₂PO₄) or mixtures thereof.

4. The oral composition for sensitive teeth of claim 1, wherein the oral composition further comprises ursodesoxycholic acid.

5. The oral composition for sensitive teeth of claim 4, wherein the amount of ursodesoxycholic acid is 0.01 to 1% by weight of the oral composition.

6. The oral composition for sensitive teeth of claim 1 or 2, wherein the oral composition further comprises an adhesive in an amount of 5 to 60% by weight of the composition, and the adhesive is precipitated silica, silica gel, zirconium silicate, dicalcium phosphate dihydrate, anhydrous dicalcium phosphate, hydrated alumina, calcium carbonate, calcium pyrophosphate, insoluble metaphosphate, aluminum silicate or mixtures thereof.

7. The oral composition for sensitive teeth of claim 1 or 2, wherein the abrasiveness ranges from 20 to 100.

8. The oral composition for sensitive teeth of claim 1 or 2, wherein the oral composition further comprises an humectant in an amount of 20 to 70% by weight of the composition, and the humectant is a concentrated glycerin (98%), glycerin (85%), a sorbitol solution (70%), xylitol; polyethylene glycols, propylene glycol, or mixtures thereof.
